(19) United States
(12) Patent Application Publication
(10) Pub. No.: US 2021/0346532 A1
(43) Pub. Date: Nov. 11, 2021

(54) System and Sanitizable Hardware for Detecting Pathogens on Hands

(71) Applicant: Kitchener Clark Wilson, Santa Barbara, CA (US)

(72) Inventor: Kitchener Clark Wilson, Santa Barbara, CA (US)

(21) Appl. No.: 17/318,910

(22) Filed: May 12, 2021

Related U.S. Application Data
(60) Provisional application No. 63/020,605, filed on May 6, 2020.

(51) Int. Cl. A61L 2/00; A61L 2/28; A61L 2/24; A61B 5/00

(52) U.S. Cl. CPC A61L 2/0094; A61L 2/0029; A61L 2/0088; A61L 2/28; A61L 2/24; A61B 2560/04; A61B 5/742; A61B 5/4848; A61L 2202/121; A61L 2202/122; A61L 2202/14; A61B 5/0064

(57) ABSTRACT

The mechanical and system design of a device to detect pathogens on hands including means to sanitize the device while protecting the user from said sanitizer emissions be they optical, gaseous or liquid.

### FIELD

This application relates generally to systems and methods for detecting pathogens on user hands, and more particularly to the hardware for making the detection procedure safe by minimizing the transfer of pathogens from one user to the next.

### BACKGROUND

Hands contaminated by pathogens are known sources for disease spread. For example, SARS-CoV-2 (Coronavirus) contagion can be spread by contaminated individual's hands coming in contact with other individuals, or indirectly by contacting surfaces that others may touch. Some of the many infections spread by direct or indirect contact are: the common cold, flu, strep throat, chicken pox, cold sores, conjunctivitis, hand-foot-and-mouth disease, molluscum contagiosum, impetigo, Ebola, polio, tetanus, and staphylococcus. Of particular interest are the Methicillin-resistant staphylococcus aureus (MRSA), that is resistant to many antibiotics, as well as many other nosocomial (hospital derived) bacteria that cause widespread patient infections (11%, and much higher if in the ICU) such as: Escherichia coli, Enterococci, and Pseudomonas aeruginosa.

Quickly detecting such pathogens without having to rely on microscopes and laboratory investigations is commonly performed using invisible benign ultra-violet light (UVA 315 to 400 nm) to trigger the pathogen to respond with a visible fluorescence (see Lewis R. Dartnell, Tom A. Roberts, Ginny Moore, John M. Ward, Jan-Peter Muller; “Fluorescence Characterization of Clinically-Important Bacteria”; PLOS ONE; September 2013, Volume 8, Issue 9, e7527). If the pathogen normally fluoresces, or is made to, then its presence can be detected visibly.

Pathogens can be made to fluoresce by exposing them to a binding antibiotic that has been adapted to include a fluorescent moiety (see Pavintorn Teratanatorn, Richard Hoskins, Thomas Swift, C. W. Ian Douglas, Joanna Shepherd, and Stephen Rimmer; “Binding of Bacteria to Poly(N-isopropylacrylamide) Modified with Vancomycin: Comparison of Behavior of Linear and Highly Branched Polymers “; Biomacromolecules 2017, 18, 2887-2899). The antibiotic attaches to the pathogen and the moiety fluoresces visibly when exposed to UVA.

Several patents and patent applications suggest the mechanics of how to build the hardware to expose and then detect pathogens using UVA. Generally, the hardware consists of an instrumented structure, open or closed, near or within which hands are placed, exposed to UVA, and the fluorescence detected. The main issue of such hardware is the likelihood that a user's hands may touch and contaminate the enclosure surfaces and thus endanger a subsequent user who may also touch them. Kanhye (US 2017/0073722) proposes an open enclosure, where hands are held under an open shelf, but does not teach decontamination. Llamido (U.S. Pat. No. 10,613,030) presents a closed box with hand openings, does teach decontamination using germicidal UVC light (200 to 280 nm); since UVC light is dangerous it must be contained, but this containment is not described.

This application teaches how to detect pathogens using UVA light, how to de-contaminate the hardware using a variety of sterilizing sources and how to contain the injurious sterilizing source emissions so as not to expose the user.

### SUMMARY

A system and detecting station to detect pathogens on hands, while making this safe, includes the following:
means to make the pathogen optically detectable;
a detecting station to report to the user the hand contamination by the detectable pathogens;
means to sanitize the detecting station without exposing the user to injurious sanitizing emissions; and
means for the user to activate and interface with the detecting station without touching any station surfaces.

The system and pathogen detecting station, here disclosed, accomplishes this in a simple and manufacturable manner.

Sanitizing uses any of the available sanitization sources including:
UVC light, the preferred embodiment as it only leaves heat when its task is completed;
liquid or gas disinfectants with the disadvantage that these must be replenished and any gas or liquid remaining after sterilization must be removed; and
intense pulsed light (see “Kinetics of Microbial Inactivation for Alternative Food Processing Technologies”; A Report of the Institute of Food Technologists for the Food and Drug Administration of the U.S. Department of Health and Human Services; submitted Mar. 29, 2000 revised Jun. 2, 2000.) that this can become practical when the requisite large high voltage power supplies are not a disadvantage to small or mobile applications.

These and other adaptable forms of sanitizing sources are here considered and included.

In one aspect the detecting station is formed as a topless inner box within an enclosing outer box. A pathogen detecting UVA lamp is attached to an inner surface of the outer box to trigger detectable fluorescence, and a sanitizing source is attached to an inner surface of the inner or outer box. When the inner box is moved toward the top of the outer box to initiate a detecting phase, a detecting space is formed where coincident cutouts in each box align to form an opening into the detecting space to allow insertion of hands for UVA activated detection. When the inner box is moved away from the top of the outer to initiate a sanitizing phase, the openings and cutouts are misaligned and the openings closed to seal the detecting space and keep sanitizing emissions from reaching the user. The inner box acts as a shutter for the sanitizing source.

In another aspect, the boxes are arranged such that the sanitizing source is also withdrawn and protected during the detecting phase, but exposed by the movement of the inner box shutter during the sanitizing phase.

Other aspects of the disclosed System and Sanitizable Hardware for Detecting Pathogens on Hands will become apparent from the following description, the drawings, and the claims.

### BRIEF DESCRIPTION OF THE DRAWINGS

FIG. 1 illustrates (a) a non-contact dispenser adapted to dispense a pathogen detecting substance including a detecting agent, and (b) is a schematic of the main features of the agent.

FIG. 2 shows the detecting station enclosure encompassing the detecting space.

FIG. 3 shows the detecting station enclosure and the inner shutter structure (here shown fully withdrawn for illustration purposes) that fits moveably within the enclosure.

FIG. 4 abc shows aspects of the inner shutter structure operation: (a) no handhole occlusion to allow hands to be inserted into the detecting space, (b) partial occlusion as the inner shutter structure is lowered, and (c) full occlusion when the inner shutter structure is lowered by at least the height of the handholes.

FIG. 5 shows, in detecting mode, how the UVA detecting lamp and the sanitizing source are arranged with the sanitizing source below the detecting space.

FIG. 6 shows, in sanitizing mode, how the sanitizing source is positioned within the sanitizing space.

FIG. 7 shows the incorporation of a control module.

FIG. 8 illustrates the construction of an LED based omnidirectional UVC sanitizing lamp.

### DETAILED DESCRIPTION

Hand Preparation: The system begins with a means to make the pathogen optically where a pathogen is an infectious microorganism or agent such as a virus, bacterium, protozoan, prion, viroid, or fungus.

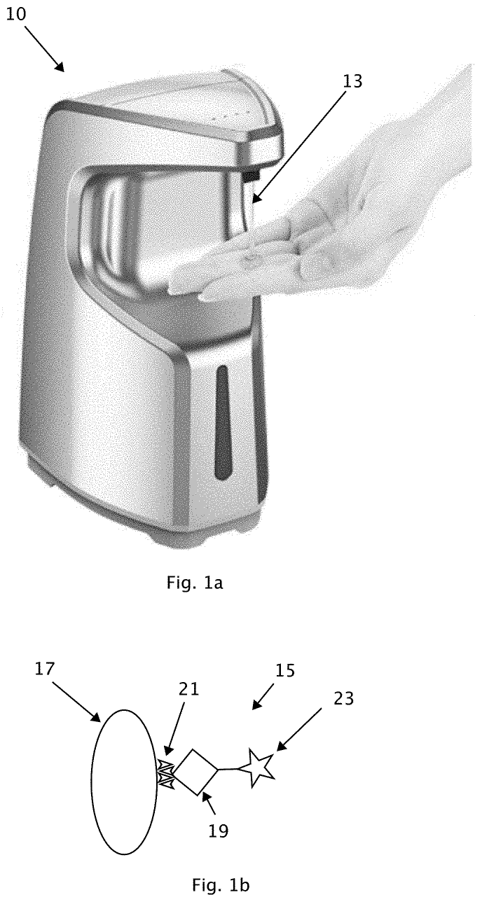

FIG. 1a shows a non-contact dispenser 10 commonly available that is adapted to dispense a detecting substance 13. The detecting substance incorporates a detecting agent 15.

FIG. 1b illustrates the detecting agent 15 that binds irreversibly to a pathogen 17 of interest, or family of pathogens, and fluoresces optically at a known wavelength under UVA light. The detecting agent 15 includes a binder component 19, such as an antibody or antibiotic or other means, responsive to the pathogen and capable of irreversibly binding 21 to the pathogen and not to items of no interest. The detecting agent further includes an UVA fluorescent signaling component 23 attached to the binder component.

In one implementation, multiple distinct detecting agents 15 responsive to distinct pathogens are combined in the dispensed detecting substance 13 to simultaneously make detectable various distinct pathogens or pathogen families.

In a second implementation, the combined multiple distinct detecting agents 15 have fluorescent signaling component 23 that fluoresce at distinct wavelengths so as to distinguish the various distinct pathogens or families.

In use, the user prepares his hands by activating the non-contact dispenser 10 to coat the hands with the dispensed detecting substance 13 and washes the hands as needed to remove unbound excess dispensed detecting substance where washing includes air drying as needed. Depending on the binder component 19, disposal of the detecting agent 15, as during hand washing, may be controlled to minimize broad dissemination and increased bacterial antibiotic resistance in the public space.

In a third implementation, the dispensed substance 13 includes a soap-like component to aid in subsequent hand washing to remove unbound detecting agent 15.

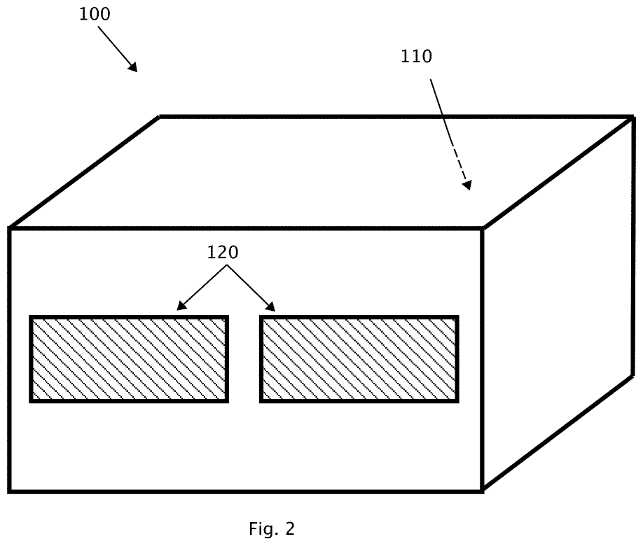

Detecting Space: FIG. 2 shows a basic detecting station enclosure 100 where the space within is the detecting space 110 where pathogen detection occurs. Hand holes 120 are provided through which the hands are inserted into the detecting space that is large enough to accommodate both (or one) hands while limiting contact with enclosure inner surfaces to minimize contamination. Two handholes are shown; in another implementation there is a single larger handhole capable of accommodating both hands. The general outline of the enclosure 100 and handholes 120 are here illustrated as boxlike, but can be of any useful shape and size.

Shuttering: A primary design issue is providing a means to sanitize the user accessed detecting space 110 without causing user harm by exposure to the sanitizing source emissions. A preferred sanitizing source is UVC light, around 250 nm wavelength, that damages the DNA or RNA of all living things, including pathogens. This either disables (kills) or eliminates their ability to reproduce. However, UVC light is equally dangerous to the user and sanitizing UVC illumination within the detecting space must have all enclosure 100 openings 120 closed (shuttered) such that UVC light does not escape the enclosure and reach the user: the handholes 120 need shutters. Shuttering is also needed when any of the other mentioned sanitizing sources are used.

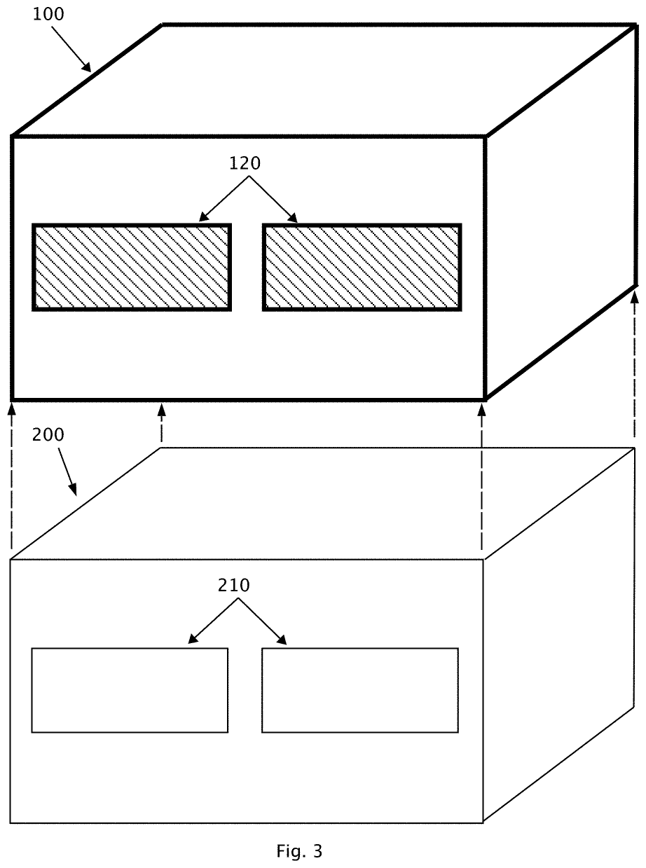

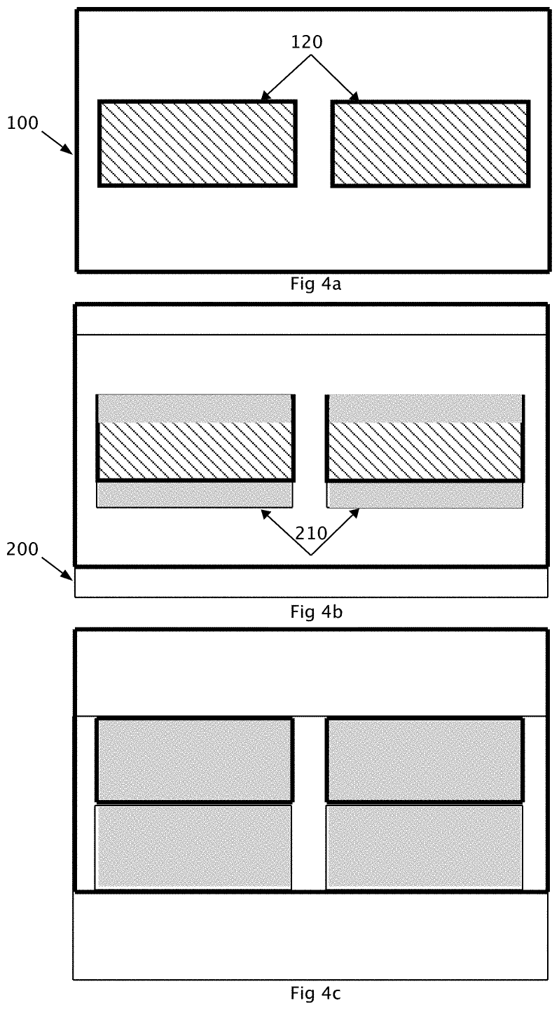

FIG. 3 shows a basic shutter formed by fitting a moveable inner shutter structure 200 within the detecting station enclosure 100. The inner shutter structure has cutouts 210 matching the handholes 120 and, when the inner structure is moved sufficiently up into the enclosure 100, the handholes and cutouts align and the user can insert his hands into the detecting space 110 for pathogen detection. When not sufficiently moved, the handholes and cutouts are misaligned and the handholes are partially or totally occluded and the detecting space 110 becomes isolated from the user for sanitizing. FIG. 4 abc illustrates the progressive misaligned and handholes shuttering.

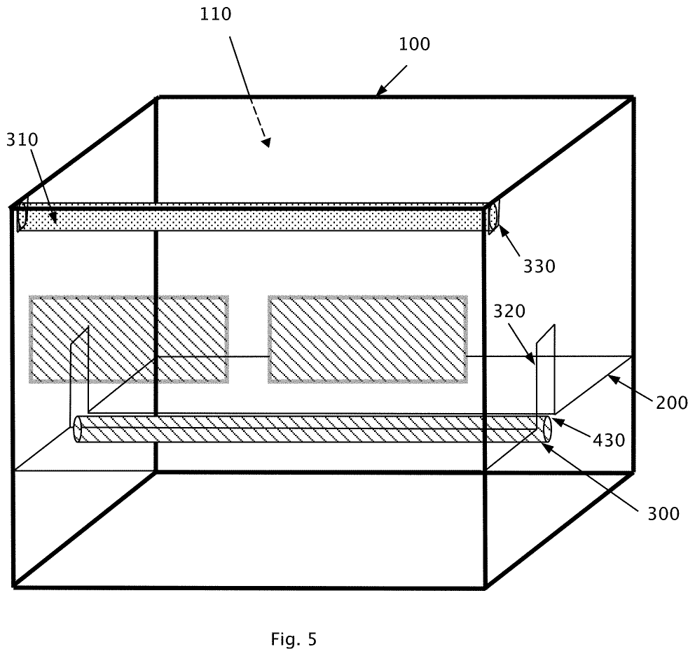

FIG. 5 illustrates the preferred detecting station embodiment where, in detecting position, the detecting station enclosure 100 handholes 120 and inner shutter structure 200 matching cutouts 210 are aligned while the sanitizing source 300 is held below the detecting space 110 to avoid the user hands. The sanitizing source is mounted through inner shutter structure sanitizing slots 320 to the enclosure walls, and the UVA lamp is mounted through inner shutter structure detecting slots 330 to the enclosure walls.

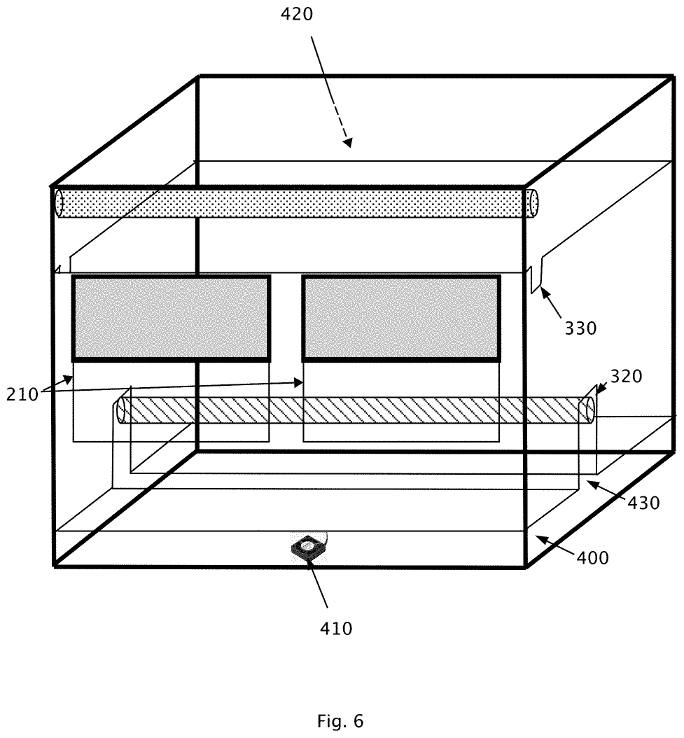

FIG. 6 illustrates the same embodiment in sanitizing position where the detecting station enclosure 100 handholes 120 and inner structure 200 matching cutouts 210 are misaligned and the sanitizing source 300 is within the larger sanitizing space 420 that includes the detecting space 110.

The enclosure 100 extends beyond the inner shutter structure 200 to make an extension space 400 for power supplies and other items. One of the items is a fan 410 to draw cooling air past the UVA detecting lamp 310 and sanitizing source 300. In detecting position, air enters from handholes. In sanitizing position, air enters from light-hooded openings (not shown) on the detecting station enclosure 100 sides that are shuttered in the same manner as the handholes, with matching cutouts in the inner shutter structure, except they are open in sanitizing position and closed in detecting position. Air is pulled into the enclosure extension space through the sanitizing source gap 430 and expelled through an opening (not shown) in the enclosure floor and then through an externally accessible N-95 filter (not shown) to minimize environmental contamination.

In one implementation, the fan 410 is left on to provide continuous positive airflow to minimize potentially contaminated outflow air.

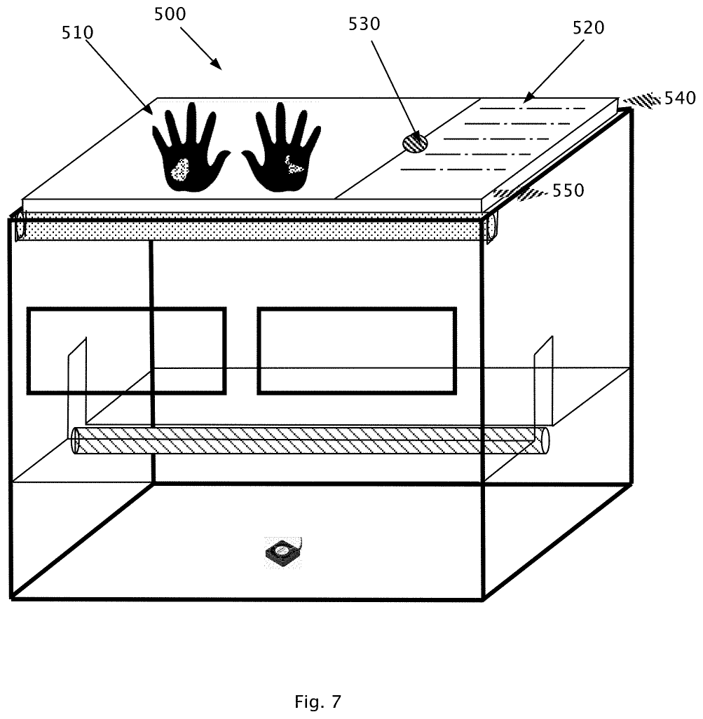

Control Module: FIG. 7 shows the addition of a control module 500. The control module includes a detecting camera (not shown) to image the detecting space 110 and an associated graphic screen. On one part of the screen is displayed the real-time image 510 of the hands with superimposed image processing enhancements indicating the location of pathogens. Another part of the screen is used to present user information and instructions 520.

As they are sources of contamination, there are no buttons or other tactile devices to control the actions of the detecting station. Rather, the control module includes a motion detector 530 for touchless user interface to sense user gestures that are interpreted as commands. This detector can be any of the optical, sonic and other sensors, or a second camera for more sophisticated gestures and to photographically identify the user.

A computer, built into the control module 500, provides station control logic and includes a communication means whereby it exchanges information with a remote device. As described below, the computer accepts inputs 540 from various sensors and generates various control signals 550.

Shutter Motion: The inner shutter structure 200 is raised, lowered and held in position using a combination of gravity, actuators, and sensors. Gravity, working on the mass of the inner shutter structure, provides force to lower it from detecting position to sanitizing position. The extent to which it is lowered is limited by physical stops (not shown) and reported to the controller 500 by a micro switch or optical or other type of position sensors. Gravity is a mild force the user can easily oppose should his hands be partially captured when the inner shutter structure is lowered.

An electric motor, with cable windup or linear actuator, raises the inner shutter structure 200 from sanitizing position to detecting position and provides speed control. Arrival at detecting position is reported by another micro switch or optical or other type of position sensor, and the inner shutter structure is held in that position by motor static tension or a solenoid actuator or other means.

Control computer operation: Normally the detecting station is in a quiescent sanitizing mode with the handholes closed and the sanitizing source off; the control module 500 is waiting for the motion detector 530 to indicate a user is ready, and provides written or audible instructions 520 for the user to make a gesture over top of the detecting station in order to initiate the pathogen detection cycle.

Initiation of the pathogen detection cycle includes activating the mechanism to raise the inner shutter structure 200 to the detecting position, and the control module 500 waits for the inner shutter structure position sensor to indicate detection position is achieved.

Upon receiving the detection position indication, the control module 500 disables the mechanism to raise the inner shutter structure, enables the mechanism to hold the inner shutter structure in detecting position, turns on the UVA detecting lamp (may always be on), begins using the detecting camera to detect and display pathogen contamination information and advice on how to improve the hand orientation, and looks for the hands to be withdrawn from the detecting station.

When the detecting camera image of the hands shows they have been withdrawn, the control module 500 terminates the detecting cycle and initiates the sanitization cycle. The inner shutter structure position holding mechanism is terminated, the inner shutter structure 200 is lowered by gravity to sanitizing position, and the control module 500 is looking for the sanitizing position indication signaling arrival at sanitizing position.

Upon receiving the sanitizing position indication, the sanitizing source 300 is turned on for a fixed sanitizing time, the cooling fan 410 is turned on (may always be on), and the control module 500 is looking for the end of the fixed sanitizing time.

At the end of the fixed sanitizing time, the preferred UVC sanitizing source 300 is turned off and the control module 500 looks for a temperature sensor indication that the fan 410 has vented sanitizing heat.

For an alternate embodiment using intense pulsed light as a sterilizing source, its generated heat is also removed by the fan 410.

For an alternate embodiment using gas as the sterilizing source, the remnant gas is purged by second fan and sequestered in an external container for disposal. The fan 410 is included to provide heat elimination and positive airflow but is not active during sterilization or when the second fan is active.

For an alternate embodiment using liquid as the sterilizing source, the remnant fluid is evacuated by a pump and sequestered in an external container for disposal. The fan 410 is included to provide heat elimination and positive airflow but is not active during sterilization or when the evacuation pump is active.

In the preferred embodiment using UVC sterilizing source, when the heat has been removed the cooling fan 410 is turned off or may be left on for continuous positive airflow.

When cleanup is completed, the sanitization cycle is terminated and the quiescent sanitizing mode is re-initiated.

It may be sufficient to perform the sanitizing cycle after every few users rather than after each one. The UVA detecting lamp 310 and sanitizing source 300 are turned on and off using pulse width modulation to vary their intensity, and the cooling fan 410 is operated in response to temperature sensors and may also be operated with pulse width modulation for airflow adjustment.

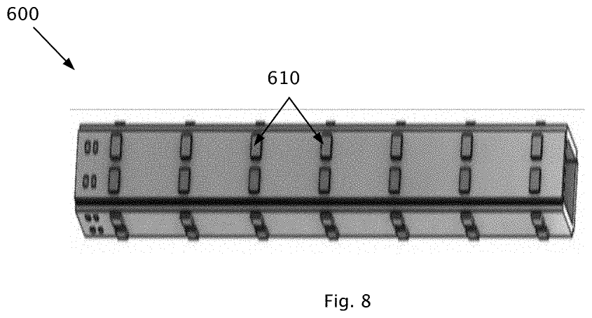

UVA and UVC lamp options: The UVA detecting light is at a user safe wavelength and may be any of a mercury or LED variety. As shown in FIG. 8, the preferred UVC sanitizing lamp 300 is formed as a square bar 600 with banks of LEDs 610 along each side. Fewer LEDs are needed if the bar is made to spin with as few as a single side of LEDs. This will require a spinner motor and electrodes, but can be less expensive than a fully populated bar. Naturally, although the bar is shown having a rectangular cross section, it can be triangular or cylindrical or any other useful shape.

Wiring: Electrical wiring and actuator cabling are routed through internal or external conduits.

Other Embodiments: While several illustrative embodiments of the invention have been shown and described, numerous variations and alternate embodiments will occur to those skilled in the art. Such variations and alternate embodiments, as well as others, are contemplated and can be made without departing from the spirit and scope of the invention as defined in the appended claims. For example:
the motion of the inner shutter structure 200 can be horizontal rather than vertical and driven entirely by motors without gravity assist;
the pathogen fluorescent wavelength may not be visible yet detectable by the detecting camera

1. A detecting station to detect UVA fluorescent pathogens on hands and having an enclosure with at least one handhold to accept at least one user hand and, further, said enclosure encompasses a moveable shutter, a UVA detecting lamp, and a sanitizing source where:
beginning with the shutter at a detecting position defining the detecting space and with at least one hand is inserted through the at least one handhole, UVA fluorescent pathogens are detected on the hand in response to emissions from said UVA detecting lamp;
with the at least one hand is removed and detection completed, the moveable shutter, in a first motion, moves to a sanitizing position forming a sanitizing space that includes the detecting space while occluding said at least one handhole and eliminating sanitizing space access external to the enclosure;
while in the sanitizing position, the sanitizing source within the sanitizing space is activated and, when sanitation is completed, the shutter, in a returning second motion, moves to the detecting position while opening the at least one handhole; and
in this manner the pathogens on said at least one hand are detected, the detection space is sanitized, and emissions of the sanitizing source are kept within the enclosure and away from the user.

2. The claim 1 first motion of the moveable shutter further acts to place the sanitizing source within the sanitizing space, and the second return motion acts to place the sanitizing source out of the detection space.

3. The claim 1 sanitizing source is a UVC lamp.

4. The claim 1 detecting station further includes a camera responsive to the pathogen fluorescence emitted in response to the UVA detecting lamp, and a control module with display to report pathogens detected by the camera to the user.

5. The control module of claim 3 further includes a touchless user interface.

6. The detecting station of claim 1 further includes a fan to draw in external air to cool the enclosure.

7. The fan of claim 5 further providing positive airflow into the enclosure to minimize potentially contaminated outflow air.
